(19) United States
(12) Patent Application Publication
(10) Pub. No.: US 2025/0134786 A1
(43) Pub. Date: May 1, 2025

(54) Composition Comprising Silicas for the Cosmetic Treatment of Keratinous Fibers

(71) Applicant: Henkel AG & Co. KGaA, Duesseldorf (DE)

(72) Inventors: Anna Puls, Winsen (DE); Marcus Noll, Quickborn (DE)

(21) Appl. No.: 18/932,959

(22) Filed: Oct. 31, 2024

(30) Foreign Application Priority Data
Oct. 31, 2023 (DE) 102023210769.4

(51) Int. Cl. A61K 8/25; A61K 8/02; A61K 8/34; A61K 8/368; A61K 8/58; A61Q 5/00; A61Q 5/06

(52) U.S. Cl. CPC A61K 8/25; A61K 8/022; A61K 8/025; A61K 8/345; A61K 8/368; A61K 8/585; A61Q 5/00; A61Q 5/06; A61K 2800/412

(57) ABSTRACT

The invention relates to a composition for the cosmetic treatment of keratinous fibers comprising a hydrophobically modified silica and a hydrophilic silica, wherein the hydrophilic silica has an average particle size d50 in the range of 6 to 18 μm, measured in accordance with ISO 13320. The invention also relates to the use of the composition for the cosmetic treatment of keratinous fibers, in particular to increase the volume of a hairstyle of keratinous fibers.

### FIELD OF INVENTION

The invention relates to a composition for the cosmetic treatment of keratinous fibers, which comprises a mixture of a hydrophilic silica with a small particle size and a hydrophobically modified silica. The composition is used in particular to increase the volume of a hairstyle of keratinous fibers.

### BACKGROUND

Compositions for the cosmetic treatment of keratinous fibers can be prepared, among other things, on the basis of silicas (amorphous silicon dioxide) as a component. Such compositions are usually in powder form. These compositions are used, for example, for nourishing and aesthetic enhancement of keratinous fibers. In particular, so-called volume powders can be produced on the basis of silicas, which give keratinous fibers increased volume and temporary deformability.

A disadvantage of known silica-based compositions is that, when used on hair, they produce a dull and rough haptic impression on the treated hair and possibly on the palms of the hands of the users. This disadvantage is particularly noticeable with long hair. In addition, known compositions occasionally produce a hair texture that is perceived as unsightly and dull.

Another property of known compositions in powder form is their more or less pronounced tendency to generate dust. Compositions that are too dry and consist of particles that are too small are more likely to produce dust when the composition is applied or removed from a container. This is disadvantageous for the ease of handling the composition.

To solve these problems, compositions based upon modified silicas have been developed in the prior art. For example, DE 10 2018 130 538 A1 discloses compositions containing a hydrophobically modified silica and particles comprising a silicone elastomer. These compositions produce sprayable volume powders to give hair a soft feel. However, the use of silicone elastomer as a component of the composition is disadvantageous, as there is a general interest in providing compositions without silicone that can be obtained from more sustainable sources and are more biodegradable.

### SUMMARY OF THE INVENTION

The invention is based upon the object of providing a cosmetic agent for the treatment of keratinous fibers. The agent is to be particularly suitable for increasing the volume of a hairstyle of keratinous fibers. Furthermore, the agent is to be easy to handle and give the fibers a pleasant, smooth feel. Finally, the agent is not to comprise any silicone-containing or poorly biodegradable components.

The invention relates to a composition for the cosmetic treatment of keratinous fibers. The composition can include a hydrophobically modified silica and a hydrophilic silica. The hydrophilic silica can have an average particle size d50 in the range of 6 to 18 μm, measured in accordance with ISO 13320. In one embodiment, the the composition may be in powder form.

The hydrophilic silica may have a substantially spherical particle shape. The hydrophilic silica may preferably have a sieve residue of less than 10%, measured on a 45 μm sieve in accordance with ISO 3262-19. In one embodiment, the hydrophobically modified silica may be a silazane-modified silica.

In a further embodiment, the composition may comprise 2 to 30 wt. % hydrophobically modified silica and 2 to 30 wt. % hydrophilic silica, in each case based upon the total weight of the composition. In still a further embodiment, the composition can further comprise an organic acid, a polyhydric alcohol, and/or a preservative.

The organic acid may preferably be a carboxylic acid having one to three carboxyl groups and 2 to 10 carbon atoms, the polyhydric alcohol may preferably have 2 to 6 carbon atoms and 2 to 3 OH groups, and/or the preservative may preferably comprise sodium benzoate. The invention also relates to a use of the composition described above for the cosmetic treatment of keratinous fibers. The composition is preferably used to increase the volume of a hairstyle of keratinous fibers and/or to improve the haptic properties of keratinous fibers.

### Advantageous Effects of the Invention

The invention provides a composition for treating keratinous fibers, which can in particular increase the volume of a hairstyle of keratinous fibers. By combining, according to the invention, a hydrophobically modified silica with a hydrophilic silica with a small particle size, the composition can be provided in an easily handled powder form, which is characterized in particular by low dust development. Furthermore, the composition not only gives the keratinous fibers a pleasant feel, but also leaves a pleasant feel on the user's hands. In accordance with the invention, all these properties can be achieved without the composition having to comprise a silicone elastomer known from the prior art. The composition according to the invention thus has the same advantageous properties as a silicone elastomer-containing composition known from the prior art, or even surpasses this. The invention does not contain any silicone-containing or poorly biodegradable components.

### DETAILED DESCRIPTION OF THE INVENTION

Hereafter, embodiments of the present invention will be described in detail. However, the present invention is not limited to the following embodiments.

The composition comprises as substantial components a hydrophobically modified and a hydrophilic silica. In the context of the invention, amorphous silicon dioxide is referred to as silica. The silicas can be produced, for example, by precipitation from a silica-containing solution (precipitated silica) or by pyrolysis of silicon-containing compounds, in particular silicon chloride (pyrogenic silica).

The combination of these components results in a composition suitable for the cosmetic treatment of keratinous fibers. In the context of the invention, cosmetic treatment of keratinous fibers refers in particular to the nourishing, shaping (in particular, temporary shaping), and creation of volume in hairstyles of keratinous fibers. In the context of the invention, keratinous fibers include in particular fur, wool, feathers, and human hair. The composition is particularly suitable for the cosmetic treatment of human or animal hair, especially for the treatment of human head hair.

The hydrophilic silica is characterized by the fact that it can be completely wetted in pure water. The hydrophilic silica can, for example, be a precipitated silica or a pyrogenic silica. Preferably, the hydrophilic silica is a precipitated silica. A mixture of hydrophilic silicas can also be used as hydrophilic silica. Preferably, the hydrophilic silica comprises no modifications with an organic component.

Preferably, it is an amorphous silica that contains no crystalline components. The crystalline proportion can be determined by X-ray diffraction and is preferably below a detection limit of 0.1 wt. %.

Of crucial importance for the invention is that the hydrophilic silica has an average particle size d50 in the range of 6 to 18 μm. The average particle size d50 is measured in accordance with ISO 13320 in the current version valid on the priority date of the invention. In particular, the average particle size d50 can be determined by means of laser diffraction.

With an average particle size d50 in the range according to the invention, the advantageous properties of the composition described above are achieved. In particular, the use of a hydrophilic silica with the specified particle size imparts to the composition the described pleasant haptic properties, in particular after drying any liquid components of the composition. At the same time, the composition retains its volume-enhancing effect on keratinous fibers. In addition, the specified particle size ensures low dust formation in the case of a powdery composition. In addition, the particles are not so large that they would be visible after application to the keratinous fibers. By using hydrophilic silica, the addition of silicone elastomers to the composition can be dispensed with. These advantages could not be achieved so far with a hydrophilic silica with a different particle size d50. The hydrophilic silica preferably has an average particle size d50 in the range of 7 to 14 μm, preferably in the range of 8 to 12 μm, most preferably an average particle size d50 in the range of 9 to 10 μm.

The hydrophilic silica preferably has a narrow particle size distribution. The particle size distribution can be determined from the volumetric sum distribution of the particle sizes measured in accordance with ISO 13320. In one embodiment, the silica has a particle size distribution characterized by the parameters d90 and d10, wherein d90 indicates the maximum diameter of 90% of the measured particles, and d10 indicates the maximum diameter of 10% of the measured particles. Preferably, d90 has a value of 60 μm or less, more preferably 50 μm or less, most preferably 40 μm or less. Preferably d10 has a value of 5 μm or less, more preferably 3 μm or less, most preferably 1 μm or less. Preferably, d90 has a value of 60 μm or less and d10 has a value of 5 μm or less, more preferably d90 has a value of 50 μm or less and d10 has a value of 3 μm or less, and most preferably d90 has a value of 40 μm or less and d10 has a value of 1 μm or less.

The hydrophilic silica preferably has a low coagulum content, i.e., a low proportion of coagulated particles. The coagulum content can be determined by sieving based upon the sieve residue of silica. Preferably, the sieve residue is determined on a 45 μm sieve in accordance with ISO 3262-19 in the current version valid on the priority date of the invention. The hydrophilic silica preferably has a sieve residue of 10% or less, more preferably 5% or less, most preferably 1% or less, each measured on a 45 μm sieve in accordance with ISO 3262-19.

The hydrophilic silica preferably consists of particles that have a substantially spherical particle shape. The particle shape can be determined using the following method. An image of a sample of silica is taken by means of scanning electron microscopy. Based upon the image, the cross-sectional area A and the circumference U of each individual particle are determined. The degree of spherical particle shape (sphericity) of the individual particles is quantified using the parameter S, wherein S is calculated from the equation S=U²/(4π A). For an ideal spherical shape, S=1. The more irregularly shaped the particle is and the more the particle shape deviates from the ideal spherical shape, the smaller S is. The sphericity of the entire sample is determined based upon the average of the sphericity of the individual particles. In one embodiment, the hydrophilic silica has a sphericity determined by this method of 0.80 or more, preferably 0.85 or more, more preferably 0.90 or more, most preferably 0.95 or more.

The hydrophilic silica preferably has a BET surface area of 24 m²/g or less, particularly preferably 18 m²/g or less, most preferably 12 m²/g or less. The BET surface area can be determined in accordance with DIN 66131 in the current version valid on the priority date of the application. Suitable hydrophilic silicas are available, for example, under the trade name Spherilex® from Evonik. The composition preferably contains 2 to 30 wt. %, preferably 3 to 20 wt. %, more preferably 5 to 15 wt. %, most preferably 6 to 12 wt. % of the hydrophilic silica, based upon the total weight of the composition. Preferably, the composition comprises at least one of the hydrophilic silicas described as preferred, preferably in the amount indicated above. The total proportion of hydrophilic silicas is in this case preferably limited to up to 30 wt. %, preferably up to 20 wt. %, particularly preferably up to 15 wt. %, most preferably up to 12 wt. %.

The hydrophobic silica is characterized by a hydrophobic surface that is not completely wetted by pure water. The hydrophobically modified silica can be obtained, for example, by modifying a precipitated silica or a pyrogenic silica. The hydrophobically modified silica is particularly preferably a pyrogenic silica. A mixture of hydrophobically modified silicas can also be used as hydrophobically modified silica.

The hydrophobically modified silica can, for example, have a hydrophobic organic coating. For example, the silica can be coated with a wax. The hydrophobically modified silica can also be obtained by modification with a siloxane, silane, or silazane compound (siloxane-, silane-, or silazane-modified silica). Such modified silicas can be used particularly well in the composition and, in combination with the hydrophilic silica described, lead to the advantages mentioned above. Alkylsiloxanes such as hexamethyldisiloxane, organosilane compounds such as dimethylsilane or trimethylsilane, or silazane compounds, e.g., hexamethyldisilazane (HDMS), are particularly suitable for modifying the silicas. However, polyorganosiloxane-modified silicas are excluded from this.

The hydrophobically modified silica is particularly preferably a silazane-modified silica, in particular a silica modified with HDMS. In one embodiment, the hydrophobically modified silica is a silazane-modified pyrogenic silica, in particular a pyrogenic silica modified with HDMS.

Preferably, the hydrophobically modified silica has a carbon content of 0.1 to 15 wt. %, preferably 1 to 10 wt. %, particularly preferably 2 to 5 wt. %, based upon the total weight of the hydrophobically modified silica.

The hydrophobically modified silica preferably has a BET surface area of 100 m²/g or more. The BET surface area is particularly preferably in the range of 100 to 300 m²/g, most preferably in the range of 150 to 250 m²/g. The BET surface area can be determined in accordance with DIN 66131 in the current version valid on the priority date of the application.

Suitable hydrophobically modified silicas are available, for example, under the trade name Aerosil® from Evonik.

The composition preferably contains 2 to 30 wt. %, preferably 3 to 20 wt. %, more preferably 5 to 15 wt. %, most preferably 6 to 12 wt. % of the hydrophobically modified silica, based upon the total weight of the composition.

Preferably, the composition comprises at least one of the hydrophobically modified silicas described as preferred, preferably in the amount indicated above. The total proportion of hydrophobically modified silicas is in this case preferably limited to up to 30 wt. %, preferably up to 20 wt. %, particularly preferably up to 15 wt. %, most preferably up to 12 wt. %.

The composition preferably comprises 2 to 30 wt. %, preferably 3 to 20 wt. %, more preferably 5 to 15 wt. %, most preferably 6 to 12 wt. % hydrophobically modified silica and 2 to 30 wt. %, preferably 3 to 20 wt. %, particularly preferably 5 to 15 wt. %, most preferably 6 to 12 wt. % of the hydrophilic silica, in each case based upon the total weight of the composition.

The composition may comprise other components. In particular, the composition may additionally comprise an organic acid, a polyhydric alcohol, and/or a preservative.

The organic acid preferably comprises a mono-, di-, or trivalent organic acid, preferably a mono-, di-, or trivalent saturated carboxylic acid. Mixtures of organic acids can also be used. More preferably, the organic acid comprises a carboxylic acid having one to three carboxyl groups and 2 to 10 carbon atoms. Most preferably, the organic acid comprises a carboxylic acid having two or three carboxyl groups and 2 to 10 carbon atoms. In one embodiment, the organic acid comprises citric acid.

The composition preferably comprises 0.05 to 0.5 wt. %, preferably 0.10 to 0.25 wt. %, particularly preferably 0.10 to 0.10 wt. % organic acid, in each case based upon the total weight of the composition. The total proportion of organic acids is preferably limited to up to 0.5 wt. %, preferably up to 0.25 wt. %, most preferably up to 0.10 wt. %, even when using one of the organic acids described as preferred.

According to the invention, the polyhydric alcohol can serve as a water-soluble nourishing agent. Furthermore, the applicability of the composition can be improved by adding a polyhydric alcohol. Preferably, the polyhydric alcohol has 2 to 6 carbon atoms and 2 to 3 OH groups. Mixtures of polyhydric alcohols can also be used. The polyhydric alcohol can, for example, be selected from the group consisting of ethylene glycol, 1,2-propylene glycol, 2-methyl-1,3-propanediol, glycerol, 1,2-butylene glycol, 1,3-butylene glycol, 1,4-butylene glycol, 1,2-pentanediol, and 1,5-pentanediol. The use of glycerol, 1,2-propanediol is particularly preferred.

The composition preferably comprises 2 to 18 wt. %, preferably 5 to 15 wt. %, particularly preferably 8 to 12 wt. % polyhydric alcohol, in each case based upon the total weight of the composition. The total proportion of polyhydric alcohols is preferably limited to up to 18 wt. %, preferably up to 15 wt. %, most preferably up to 12 wt. %, even when using one of the polyhydric alcohols described as preferred.

The preservative preferably comprises at least one bacteriostatic or fungistatic compound. Mixtures of different preservatives can also be used. Preferably, the preservative comprises sodium benzoate. The composition preferably comprises 0.1 to 1 wt. % preservative, based upon the total weight of the composition. The total proportion of preservatives is preferably limited to up to 1 wt. %, even when using one of the preservatives described as preferred.

Furthermore, the composition may comprise other auxiliaries which are typically contained in compositions for the treatment of keratinous fibers, in particular in volume powders.

For example, the composition may comprise a fragrance (perfume). The fragrance may contain a mixture of different fragrances. The amount of fragrance is preferably limited to 0.01 to 1 wt. %, based upon the total weight of the composition.

The composition may further comprise plant extracts. For example, the composition may contain a coconut extract. The plant extracts are preferably aqueous and may contain small amounts of alcohol. The amount of plant extract is preferably limited to 0.01 to 5 wt. %, preferably 0.1 to 2 wt. %, based upon the total weight of the composition.

The composition preferably comprises no or only small amounts of silicone elastomers. In the context of the invention, silicone elastomers are referred to in particular as polyorganosiloxanes, in particular cross-linked polyorganosiloxanes, in particular polyorganosiloxanes with more than three siloxane units. Preferably, the amount of silicone elastomers and/or the amount of polyorganosiloxanes having more than three siloxane units is limited to 5 wt. % or less, preferably 1 wt. % or less, most preferably 0.1 wt. % or less, in each case based upon the total weight of the composition.

The composition preferably comprises water, preferably in an amount of from 40 to 96 wt. %, more preferably from 60 to 94 wt. %, further preferably from 65 to 90 wt. %, most preferably from 70 to 88 wt. %, in each case based upon the total weight of the composition.

The composition is preferably in powder form. Preferably, a powdery composition has a particle diameter determined by sieving of 0.5 mm or less, preferably 0.1 mm or less.

In one embodiment, the composition comprises a silazane-modified silica, a hydrophilic silica having an average particle size d50 in the range of 6 to 18 μm, a polyhydric alcohol, a preservative, and an organic acid.

In one embodiment, the composition comprises a silazane-modified silica having a BET surface area in the range of 100 to 300 m²/g, a hydrophilic silica having an average particle size d50 in the range of 6 to 18 μm, a polyhydric alcohol, a preservative, and an organic acid.

In one embodiment, the composition comprises an HDMS-modified silica having a BET surface area in the range of 100 to 300 m²/g, a hydrophilic silica having an average particle size d50 in the range of 6 to 18 μm, a polyhydric alcohol, a preservative, and an organic acid.

In one embodiment, the composition comprises a silazane-modified silica, a hydrophilic silica having an average particle size d50 in the range of 6 to 18 μm, an average particle size d90 of 60 μm or less, and an average particle size d10 of 5 μm or less, a polyhydric alcohol, a preservative, and an organic acid.

In one embodiment, the composition comprises a silazane-modified silica having a BET surface area in the range of 100 to 300 m²/g, a hydrophilic silica having an average particle size d50 in the range of 6 to 18 μm, an average particle size d90 of 60 μm or less, and an average particle size d10 of 5 μm or less, a polyhydric alcohol, a preservative, and an organic acid.

In one embodiment, the composition comprises an HDMS-modified silica having a BET surface area in the range of 100 to 300 m²/g, a hydrophilic silica having an average particle size d50 in the range of 6 to 18 μm, an average particle size d90 of 60 μm or less, and an average particle size d10 of 5 μm or less, a polyhydric alcohol, a preservative, and an organic acid.

In one embodiment, the composition comprises a silazane-modified silica, a hydrophilic silica having an average particle size d50 in the range of 6 to 18 μm and a sphericity of 0.90 or more, a polyhydric alcohol, a preservative, and an organic acid.

In one embodiment, the composition comprises a silazane-modified silica having a BET surface area in the range of 100 to 300 m²/g, a hydrophilic silica having an average particle size d50 in the range of 6 to 18 μm and a sphericity of 0.90 or more, a polyhydric alcohol, a preservative, and an organic acid.

In one embodiment, the composition comprises an HDMS-modified silica having a BET surface area in the range of 100 to 300 m²/g, a hydrophilic silica having an average particle size d50 in the range of 6 to 18 μm and a sphericity of 0.90 or more, a polyhydric alcohol, a preservative, and an organic acid.

In one embodiment, the composition comprises a silazane-modified silica, a hydrophilic silica having an average particle size d50 in the range of 6 to 18 μm and a BET surface area of 24 m²/g or less, a polyhydric alcohol, a preservative, and an organic acid.

In one embodiment, the composition comprises a silazane-modified silica having a BET surface area in the range of 100 to 300 m²/g, a hydrophilic silica having an average particle size d50 in the range of 6 to 18 μm and a BET surface area of 24 m²/g or less, a polyhydric alcohol, a preservative, and an organic acid.

In one embodiment, the composition comprises a HDMS-modified silica having a BET surface area in the range of 100 to 300 m²/g, a hydrophilic silica having an average particle size d50 in the range of 6 to 18 μm and a BET surface area of 24 m²/g or less, a polyhydric alcohol, a preservative, and an organic acid.

In one embodiment, the composition comprises:
5 to 20 wt. % polyhydric alcohol,
0.1 to 1 wt. % preservative,
0.05 to 0.5 wt. % organic acid,
5 to 15 wt. % hydrophilic silica with an average particle size d50 in the range of
6 to 18 μm,
5 to 15 wt. % silazane-modified silica,
0.1 to 1 wt. % fragrances, and
60 to 85 wt. % water,
   in each case based upon the total weight of the composition.

In one embodiment, the composition comprises
5 to 20 wt. % glycerol,
0.1 to 1 wt. % preservative,
0.05 to 0.5 wt. % citric acid,
5 to 15 wt. % hydrophilic precipitated silica with an average particle size d50 in the range of 6 to 18 μm,
5 to 15 wt. % HDMS-modified pyrogenic silica,
0.1 to 1 wt. % fragrances, and
60 to 85 wt. % water,
   in each case based upon the total weight of the composition.

According to the invention, the composition is used for the cosmetic treatment of keratinous fibers. In particular, the composition is used for the cosmetic treatment of human hair. Preferably, the composition is used for shaping keratinous fibers, for texturing keratinous fibers, for increasing the volume of a hairstyle of keratinous fibers, or for improving the surface properties, in particular the haptic properties, of keratinous fibers. For example, the composition can be used to give keratinous fibers, in particular human hair, a pleasant, smooth feel. In a preferred embodiment, the composition is used to increase the volume of a hairstyle of keratinous fibers, in particular a human hair hairstyle.

The composition is preferably used in powder form.

When used, the composition can be applied to the keratinous fibers, for example, with the hands or by spraying. The composition is particularly preferably applied by spraying.

In one embodiment, the invention also comprises a spray applicator containing the composition. The spray applicator has a container for holding the composition and a mechanism for spraying the composition. Preferably, the spray applicator contains the composition in powder form. Preferably, the spray applicator comprises a pumping mechanism that agitates and sprays the composition by generating an air stream. The use of a propellant can therefore be dispensed with.

### EXAMPLES

The present invention will now be described in more detail by way of examples. However, the present invention is not limited to the following examples.

A composition according to the invention was prepared according to the following recipe by mixing the indicated components.

| Component | Wt. % |
|---|---|
| Glycerol (99.5%) | 10.0 |
| Coconut extract | 1.0 |
| Sea salt | 0.2 |
| Sodium benzoate | 0.4 |
| Citric acid monohydrate | 0.12 |
| Aerosil ® R 812 S (HDMS-modified pyrogenic silica) | 7.5 |
| Spherilex ® 10 PC (hydrophilic silica with d50 = 8.0 to | 7.5 |
| 12.0 μm) | |
| Fragrance composition | 0.3 |
| Water | 72.98 |

The result was a powdery composition that could be massaged into hair by hand without creating dust. The hair could then be styled into the desired shape. The composition has a volume-enhancing effect on the hair and leaves a pleasant haptic impression on hair and hands.

What is claimed is:

1. A composition for the cosmetic treatment of keratinous fibers, comprising:
a hydrophobically modified silica and
a hydrophilic silica,
wherein the hydrophilic silica has an average particle size d50 in the range of 6 to 18 μm, measured in accordance with ISO 13320.

2. The composition according to claim 1, wherein the composition is in powder form.

3. The composition according to claim 1, wherein the hydrophilic silica has a substantially spherical particle shape.

4. The composition according to claim 1, wherein the hydrophilic silica has a sieve residue of less than 10%, measured on a 45 μm sieve in accordance with ISO 3262-19.

5. The composition according to claim 1, wherein the hydrophobically modified silica is a silazane-modified silica.

6. The composition according to claim 1, wherein the composition comprises
2 to 30 wt. % hydrophobically modified silica and
2 to 30 wt. % hydrophilic silica,
in each case based upon the total weight of the composition.

7. The composition according to claim 1, wherein the composition further comprises an organic acid,
a polyhydric alcohol, and
a preservative.

8. The composition according to claim 7, wherein
the organic acid is a carboxylic acid containing one to three carboxyl groups and 2 to 10 carbon atoms,
the polyhydric alcohol has 2 to 6 carbon atoms and 2 to 3 OH groups, and
the preservative comprises sodium benzoate.

9. A method of cosmetically treating kerationous fibers, comprising applying a composition according to claim 1 to keratinous fibers.

10. The method according to claim 9, wherein the composition is used to increase the volume of a hairstyle of keratinous fibers and/or to improve the haptic properties of keratinous fibers.
